(19) United States
(12) Patent Application Publication
(10) Pub. No.: US 2014/0296079 A1
(43) Pub. Date: Oct. 2, 2014

(54) METHODS FOR EARLY DETECTION OF ESOPHAGEAL CANCER

(71) Applicant: NEOGENOMICS LABORATORIES, Fort Myers, FL (US)

(72) Inventor: Maher Albitar, Valley Center, CA (US)

(73) Assignee: NEOGENOMICS LABORATORIES, Fort Myers, FL (US)

(21) Appl. No.: 14/096,003

(22) Filed: Dec. 3, 2013

Related U.S. Application Data
(60) Provisional application No. 61/732,618, filed on Dec. 3, 2012.

(51) Int. Cl. C12Q 1/68

(52) U.S. Cl. CPC C12Q 1/6886; G06F 19/22
USPC 506/2; 506/9; 702/20

(57) ABSTRACT

The present invention relates to a method for screening, predicting or prognosing esophageal adenocarcinoma/high grade dysplasia in a subject.

### RELATED APPLICATIONS

This application claims the benefit of the priority of U.S. provisional application No. 61/732,618, filed Dec. 3, 2012.

### FIELD OF THE INVENTION

The present invention relates to a method for screening, predicting or prognosing esophageal adenocarcinoma/high grade dysplasia in a subject.

### BACKGROUND OF THE INVENTION

Esophageal adenocarcinoma (EAC) is a deadly cancer with five-year survival rate around 17%. The incidence of esophageal cancer is increasing at a dramatic rate in the United States. The development of esophageal adenocarcinoma (EAC), in the vast majority of instances, occurs as a result of a pre-cancerous condition of the esophagus, called Barrett's Esophagus, which carries up to a 60-fold increased risk of developing EAC.

Barrett's esophagus (BE) is a preneoplastic condition in which the squamous epithelium of the distal esophagus undergoes transformation to intestinal metaplasia (IM). It is estimated to affect 10% to 12% of patients with chronic gastroesophageal reflux disease, the most significant known risk factor in the development of BE, as well as 6% of those without symptoms. Patients with BE are approximately 125 times more likely to develop carcinoma than the general population. The evolution to cancer from Barrett's epithelium is generally accepted to be a multistep progression as follows: (1) IM without dysplasia, (2) low-grade dysplasia (LGD), (3) high-grade dysplasia (HGD), and (4) esophageal adenocarcinoma (EA).

BE is caused by long-standing or severe cases of Gastro-esophageal Reflux Disorder, more commonly known as GERD, in which stomach acid repeatedly refluxes up into the lower portion of the esophagus damaging and inflaming the tissue. The constant exposure to acid and inflammatory damage may cause permanent genomic abnormalities in individual dividing cells. These genomic abnormalities in the appropriate combination may eventually lead to overt cancerous growth. However, frequently prior to overt cancer, various degrees of morphologic and genetic abnormalities can be seen and these morphologically manifest as low or high grade dysplasia. Prior to dysplasia, the normal esophagus squamous epithelium is replaced by an abnormal columnar epithelium, called specialized intestinal metaplasia in patients with BE.

The most common symptom of GERD is heartburn, although people may also experience extra-esophageal symptoms from reflux like chronic cough, chronic sore throat, allergy or flu like symptoms, even sleep apnea. In many cases, there may be no symptoms at all. Patients diagnosed with heartburn symptoms, who also have other risk factors, are typically referred for further evaluation by a gastroenterologist. Patients diagnosed with extra-esophageal symptoms, such as chronic cough or sore throat, are typically referred for further evaluation by an otolaryngologist or ear, nose, and throat (ENT) physician.

The American College of Gastroenterology recommends that people diagnosed with BE should be monitored for the development of dysplasia and EAC by undergoing regular endoscopic examinations of the esophagus and obtaining four-quadrant biopsies at 1 to 2 cm intervals of the affected esophagus. Recommended follow-up intervals are based on the absence, or presence, of dysplasia and if present, the degree of dysplasia. Frequently, dysplasia progresses or regresses due to acid suppression therapy. So it is important to adjust follow-up intervals based on recent changes in status until confirmed.

However, dysplasia evaluation is subjective and varies significantly dependent on the pathologist experience and the provided samples. Furthermore, sampling can be very serious limiting factor and despite the multiple biopsy sampling, a lesion could be easily missed. It has been suggested that brushing of the esophageal mucosa may be more representative. However, morphologic evaluation of the cytologic sample from brushing is very difficult when the goal is to determine the level of dysplasia.

Studies suggested that fluorescent in situ hybridization (FISH) testing can help distinguish cancer or high grade dysplasia from normal or low grade dysplasia. Work by Fritcher et al. assessed the relative sensitivity and specificity of conventional cytology, DNA ploidy analysis with digital image analysis (DIA), and fluorescence in situ hybridization (FISH) for the detection of dysplasia and adenocarcinoma in endoscopic brushing specimens from 92 patients undergoing endoscopic surveillance for BE. See, Fritcher et al., Human Pathology, 2008 August; 39(8): 1128-1135.

The findings from this study suggest that FISH has high sensitivity for the detection of dysplasia and EA in BE patients, with the power to stratify patients by FISH abnormality for progression to HGD/EA. Using FISH, a greater number of chromosomal alterations were detected as the severity of histologic diagnosis increased from intestinal metaplasia to adenocarcinoma. See, Brankley, et al., Human Pathology, 2012 February; 43(2):172-179. Four genes have been identified as being statistically significant in the progression from BE to HGD/EA include: p16 gene (9p21), HER2 gene (17q12), MYC gene (8q24), and ZFN 217 gene (20q13.2). Preliminary tests based on these genes have been reported but additional work is still needed to develop a clinical test that can interpret the relative importance of the data generated during the test to quickly and reliably render a diagnostic decision.

Therefore, a reliable method for early detection of esophageal cancer is crucial but has yet to be discovered. Provided herein, is a solution to this unmet need.

### SUMMARY

In various embodiments, provided herein is a method for the early detection of cancerous changes in the esophagus. This method takes advantage of performing pan-brushing of the esophageal mucosa (thorough sampling) and performing very sensitive high throughput genomic analysis using Next Generation sequencing (objective and sensitive testing). The detection of mutations in driver genes in the brushing samples is reliable objective method for early detection of EAC.

In some embodiments, the use of next generation sequencing (NGS) in a combination with brushing approach for screening for the presence of high grade dysplasia or EAC in patients with BE. In these embodiments, sampling by brushing allows surveying of the entire esophagus and the NGS allows testing of a small DNA sample for mutations in numerous oncogenes and tumor suppressor genes.

In various embodiments, provided herein is a method for identifying esophageal adenocarcinoma (EAC) in a subject comprising: pan-brushing the esophageal mucosa of the subject to obtain a sample; isolating DNA from the sample; performing next generation sequencing (NGS) on the isolated DNA; and analyzing the sequencing results and identifying any genomic abnormalities in one or more driver genes, wherein an abnormality indicates the subject has esophageal adenocarcinoma. In specific embodiments, the entire esophageal mucosa is pan-brushed.

In some embodiments the one or more driver genes are oncogenes or tumor suppressor genes. In specific embodiments the one or more driver genes are selected from the group comprising:

| | | | |
|---|---|---|---|
| ABL1 | EGFR | GNAS | MLH1 |
| SEQ ID | SEQ ID | SEQ ID | SEQ ID |
| NO. 1 | NO. 2 | NO. 3 | NO. 4 |
| RET | AKT1 | ERBB2 | CSF1R |
| SEQ ID | SEQ ID | SEQ ID | SEQ ID |
| NO. 5 | NO. 6 | NO. 7 | NO. 8 |
| HNF1A | MPL | SMAD4 | ALK |
| SEQ ID | SEQ ID | SEQ ID | SEQ ID |
| NO. 9 | NO. 10 | NO. 11 | NO. 12 |
| ERBB4 | HRAS | NOTCH1 | GNAQ |
| SEQ ID | SEQ ID | SEQ ID | SEQ ID |
| NO. 13 | NO. 14 | NO. 15 | NO. 16 |
| SMARCB1 | APC | FBXW7 | IDH1 |
| SEQ ID | SEQ ID | SEQ ID | SEQ ID |
| NO. 17 | NO. 18 | NO. 19 | NO. 20 |
| ATM | FGFR1 | MET | JAK2 |
| SEQ ID | SEQ ID | SEQ ID | SEQ ID |
| NO. 21 | NO. 22 | NO. 23 | NO. 24 |
| BRAF | FGFR2 | JAK3 | PDGFRA |
| SEQ ID | SEQ ID | SEQ ID | SEQ ID |
| NO. 25 | NO. 26 | NO. 27 | NO. 28 |
| STK11 | RB1 | CDH1 | FGFR3 |
| SEQ ID | SEQ ID | SEQ ID | SEQ ID |
| NO. 29 | NO. 30 | NO. 31 | NO. 32 |
| KDR | PIK3CA | TP53 | CDKN2A |
| SEQ ID | SEQ ID | SEQ ID | SEQ ID |
| NO. 33 | NO. 34 | NO. 35 | NO. 36 |
| FLT3 | KIT | PTEN | VHL |
| SEQ ID | SEQ ID | SEQ ID | SEQ ID |
| NO. 37 | NO. 38 | NO. 39 | NO. 40 |
| PTPN11 | CTNNB1 | KRAS | GNA11 |
| SEQ ID | SEQ ID | SEQ ID | SEQ ID |
| NO. 41 | NO. 42 | NO. 43 | NO. 44 |
| NRAS | SRC | NPM1 | SMO |
| SEQ ID | SEQ ID | SEQ ID | SEQ ID |
| NO. 45 | NO. 46 | NO. 47 | NO. 48 |

In some embodiments one or more of these genes are completely sequenced and analyzed. In other embodiments, all of these genes are completely sequenced and analyzed.

In some embodiments, an abnormality in at least one gene indicates the subject has esophageal adenocarcinoma. In other embodiment an abnormality in 2-8 genes indicates the subject has esophageal adenocarcinoma.

In specific embodiments, the sequencing is step is performed using the ILLUMINA® MISEQ® sequencer, or an equivalent thereto. In some embodiments, the analysis is performed using multiple filters.

In some embodiments, 5% prevalence for the abnormality indicates the subject has esophageal adenocarcinoma. In other embodiments, 10% prevalence for the abnormality indicates the subject has esophageal adenocarcinoma.

In one embodiment, a method for determining the risk of having HGD/Esophageal cancer, including the steps of: performing fluorescence in situ hybridization (FISH) testing on esophageal biopsy samples taken from a patient known or suspected to have Barrett's esophagus (BE); generating values corresponding to each of a plurality of data points obtained during FISH testing, the plurality of data points including data selected from the group consisting of average signals of each of four probes (MYC, 9p21, HER2, and ZNF217), percentage of cells with multiple gains, percentage of cells with single gains, percentage of cells with 9p21 hemizygous deletion, percentage of cells with 9p21 homozygous deletion, percentage of cells with tetrasomy, and percentage of cells with gains and deletions; applying an algorithm to the values to generate a numerical score; comparing the numerical score to one or more thresholds, wherein the threshold defines a distinction between HGD/EC and normal or non-HGD/EC. In various embodiments, the one or more thresholds comprise:

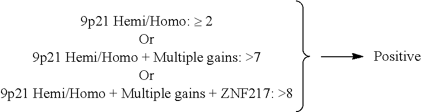

In another aspect of the invention, a method is provided for morphologic evaluation of BE histology, including the steps of performing fluorescence in situ hybridization (FISH) testing on esophageal biopsy samples taken from a patient known or suspected to have Barrett's esophagus (BE); generating values corresponding to each of a plurality of data points obtained during FISH testing, the plurality of data points including data selected from the group consisting of average signals of each of four probes (MYC, 9p21, HER2, and ZNF217), percentage of cells with multiple gains, percentage of cells with single gains, percentage of cells with 9p21 hemizygous deletion, percentage of cells with 9p21 homozygous deletion, percentage of cells with tetrasomy, and percentage of cells with gains and deletions; applying an algorithm to the values to generate a numerical score; comparing the numerical score to one or more thresholds, wherein the threshold defines a distinction between HGD/EC and normal or non-HGD/EC.

In still another aspect of the invention, a method is provided for determining the prognosis of BE, including the steps of performing fluorescence in situ hybridization (FISH) testing on esophageal biopsy samples taken from a patient known or suspected to have Barrett's esophagus (BE); generating values corresponding to each of a plurality of data points obtained during FISH testing, the plurality of data points including data selected from the group consisting of average signals of each of four probes (MYC, 9p21, HER2, and ZNF217), percentage of cells with multiple gains, percentage of cells with single gains, percentage of cells with 9p21 hemizygous deletion, percentage of cells with 9p21 homozygous deletion, percentage of cells with tetrasomy, and percentage of cells with gains and deletions; applying an algorithm to the values to generate a numerical score; comparing the numerical score to one or more thresholds, wherein the threshold defines a distinction between HGD/EC and normal or non-HGD/EC.

In various embodiments, provided herein is a method is provided for classifying cancer/HGD vs. normal, intestinal metaplasia, or normal using a FISH test based on the four genes: p16 gene (9p21), HER2 gene (17q12), MYC gene (8q24), and ZFN 217 gene (20q13.2). A step-wise algorithm is used to interpret ten different variables obtained from the FISH test to generate a classification for diagnosis. The algorithm represents a tool for putting each of these variables into its proper context for providing a diagnosis with a high degree of sensitivity. In some embodiments, the testing sensitivity is about 86%. In other embodiments, the testing sensitivity is about 67%. In various embodiments, the following algorithm is used to apply thresholds to variable values to generate a positive or negative indication:

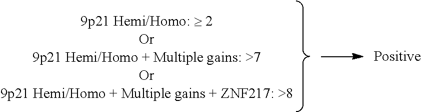

In one aspect of the invention, a method if provided for determining the risk of having HGD/Esophageal cancer, including the steps of: performing fluorescence in situ hybridization (FISH) testing on esophageal biopsy samples taken from a patient known or suspected to have Barrett's esophagus (BE); generating values corresponding to each of a plurality of data points obtained during FISH testing, the plurality of data points including data selected from the group consisting of average signals of each of four probes (MYC, 9p21, HER2, and ZNF217), percentage of cells with multiple gains, percentage of cells with single gains, percentage of cells with 9p21 hemizygous deletion, percentage of cells with 9p21 homozygous deletion, percentage of cells with tetrasomy, and percentage of cells with gains and deletions; applying an algorithm to the values to generate a numerical score; comparing the numerical score to one or more thresholds, wherein the threshold defines a distinction between HGD/EC and normal or non-HGD/EC. In these embodiments, the one or more thresholds comprise:

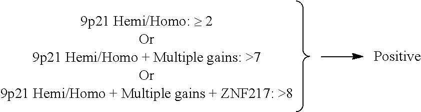

and a positive result indicates a high risk of having High Grade Dysplasia (HGD)/Esophageal Adenocardinoma (EAC) and a negative result indicates a low risk of having HGD/EAC and is suggestive or normal, reactive, or low grade dysplasia.

In another aspect of the invention, a method is provided for morphologic evaluation of BE histology, including the steps of performing fluorescence in situ hybridization (FISH) testing on esophageal biopsy samples taken from a patient known or suspected to have Barrett's esophagus (BE); generating values corresponding to each of a plurality of data points obtained during FISH testing, the plurality of data points including data selected from the group consisting of average signals of each of four probes (MYC, 9p21, HER2, and ZNF217), percentage of cells with multiple gains, percentage of cells with single gains, percentage of cells with 9p21 hemizygous deletion, percentage of cells with 9p21 homozygous deletion, percentage of cells with tetrasomy, and percentage of cells with gains and deletions; applying an algorithm to the values to generate a numerical score; comparing the numerical score to one or more thresholds, wherein the threshold defines a distinction between HGD/EC and normal or non-HGD/EC.

In still another aspect of the invention, a method is provided for determining the prognosis of BE, including the steps of performing fluorescence in situ hybridization (FISH) testing on esophageal biopsy samples taken from a patient known or suspected to have Barrett's esophagus (BE); generating values corresponding to each of a plurality of data points obtained during FISH testing, the plurality of data points including data selected from the group consisting of average signals of each of four probes (MYC, 9p21, HER2, and ZNF217), percentage of cells with multiple gains, percentage of cells with single gains, percentage of cells with 9p21 hemizygous deletion, percentage of cells with 9p21 homozygous deletion, percentage of cells with tetrasomy, and percentage of cells with gains and deletions; applying an algorithm to the values to generate a numerical score; comparing the numerical score to one or more thresholds, wherein the threshold defines a distinction between HGD/EC and normal or non-HGD/EC.

### BRIEF DESCRIPTION OF THE DRAWINGS

FIG. 1 is an illustrative tile blot showing the results of next generation sequencing gene by gene according to one embodiment.

FIG. 2A is a table listing the Barrett's FISH probe set according to one embodiment.

FIG. 2B is a sample image of a FISH signal pattern of normal esophageal cells with 2 signals of each of 4 probes of FIG. 2A: MYC, HER2, p16 and ZNF217.

FIG. 2C is a sample image of a FISH signal pattern of a normal cell with 2 signals of each of 4 probes.

FIG. 2D is a sample image of a FISH signal pattern of an abnormal cell with varying numbers of signals of each of 4 probes. (Note that not all signals of each probe are labeled.)

FIG. 2E is a sample image of a FISH signal pattern in normal tissue. Although not all visible or individually labeled, it can be seen that the numbers of signals for each of the probes are relatively uniform.

FIG. 2F is a sample image of a FISH signal pattern in abnormal tissue, where individual cells in the tissue exhibit many signals for several of the probes.

FIG. 3 provides the %9p21 Hemizygous+%9p21 Homozygous in N-LGD and HGD/CA according to one embodiment. Cut-off≧2, sensitivity=0.79, specificity=0.06.

FIG. 4 provides the %9p21 Hemizygous+%9p21 Homozygous+% Multiple gains in N-LGD and HGD/CA according to one embodiment. Cut-off≧3, sensitivity=0.86, specificity=0.44 and Cut-off≧7, sensitivity=0.57, specificity=0.88.

FIG. 5 provides the %9p21 Hemizygous+%9p21 Homozygous+% Multiple gains+ZNF217 signals (N−2) in N-LGD and HGD/CA according to one embodiment. Cut-off≧8, sensitivity=0.57, specificity=0.88.

### DETAILED DESCRIPTION OF THE INVENTION

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

All patents and publications referred to herein are incorporated by reference in their entirety.

### Certain Exemplary Terminology

Unless defined otherwise, all technical and scientific terms used herein have the same meaning as is commonly understood by one of skill in the art to which the claimed subject matter belongs. In the event that there is a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless otherwise stated. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the appended claims define the scope of the invention and that methods and structures within the scope of these claims and their equivalents be covered thereby. Abbreviations used herein have their conventional meaning within the chemical and biological arts.

Unless defined otherwise, all technical and scientific terms used herein have the same meaning as is commonly understood by one of skill in the art to which this invention belongs.

All patents and publications referred to herein are incorporated by reference.

Having now generally described the invention, the same will be more readily understood through reference to the following examples which are provided by way of illustration, and are not intended to be limiting of the present invention, unless specified.

### EXAMPLES

### Example 1

### Ancillary Four-Probe FISH Test for Diagnosis of Esophageal Cancer

A FISH test based on the four genes: p16 gene (9p21) (SEQ ID NO. 49; Accession #AB049820), HER2 gene (17q12) (SEQ ID NO. 50; Accession # NM_{—}131089), MYC gene (8q24 (SEQ ID NO. 51; Accession #M16261), and ZEN 217 gene (20q13.2) (SEQ ID NO. 52; Accession #NM_{—}006526) was performed to classify cancer (high grade dysplasia/adenocarcinoma) vs. normal or intestinal metaplasia cells.

A step-wise algorithm was used to interpret ten different variables obtained from the FISH test to generate a classification for diagnosis. The algorithm was shown to represent a tool for putting each of these variables into its proper context for providing a diagnosis with a high degree of sensitivity (86%). The sensitivity in initial testing was 67%.

The algorithm used applies thresholds to variable values to generate a positive or negative indication was:

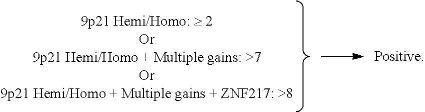

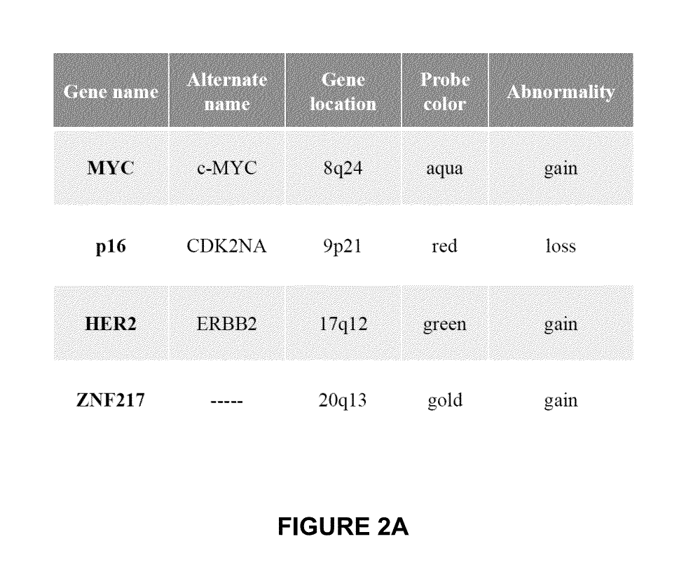

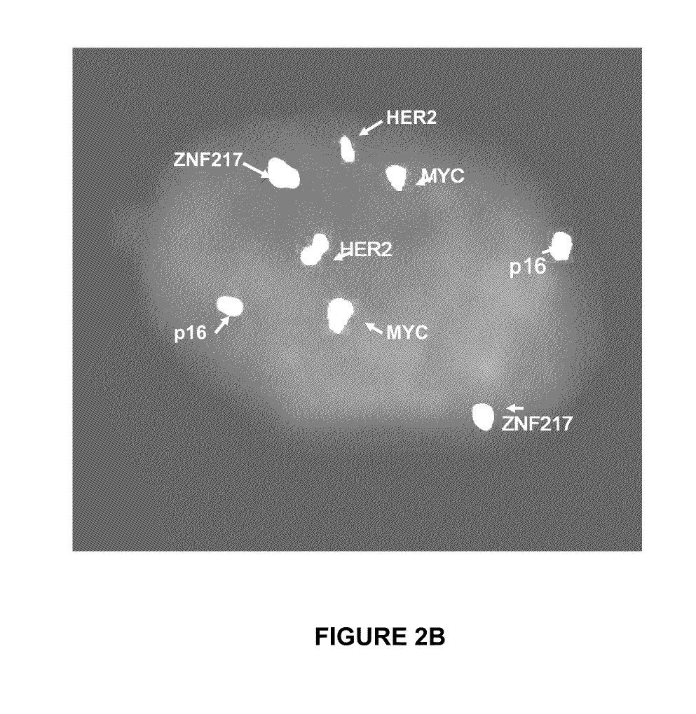

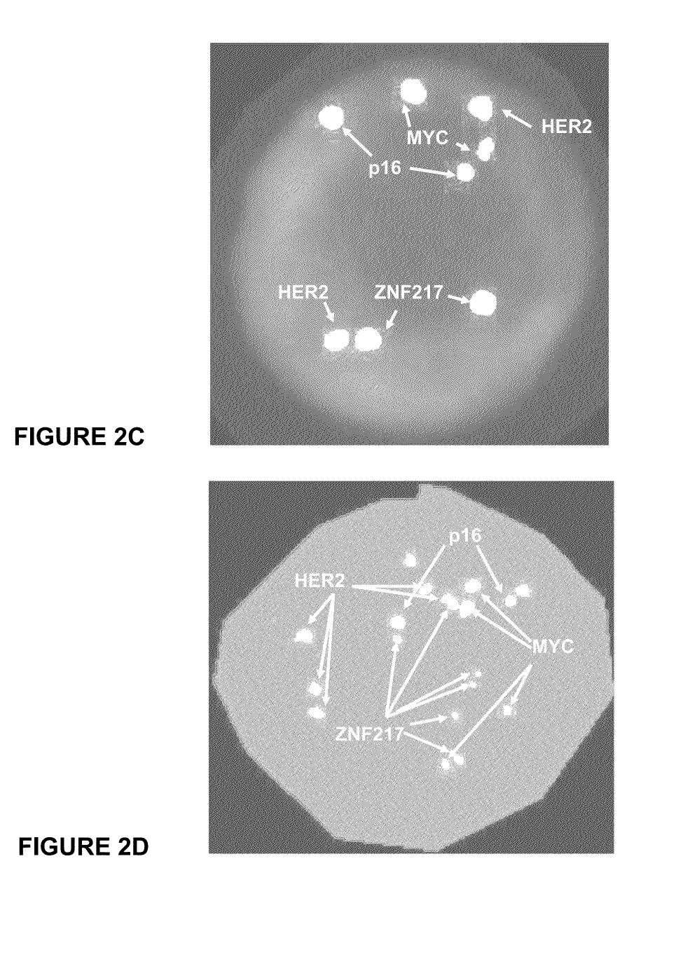

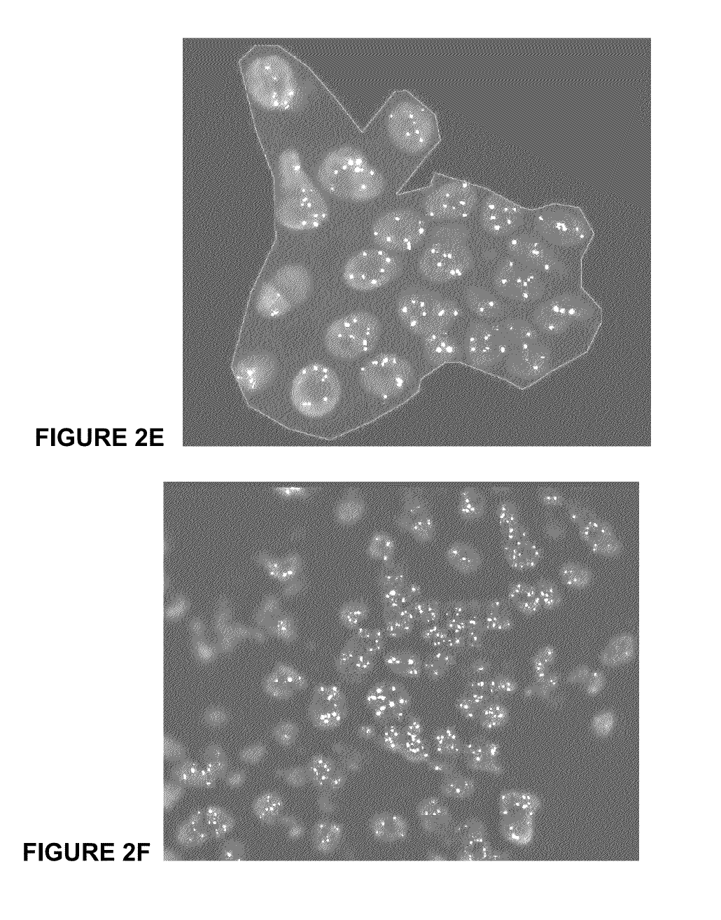

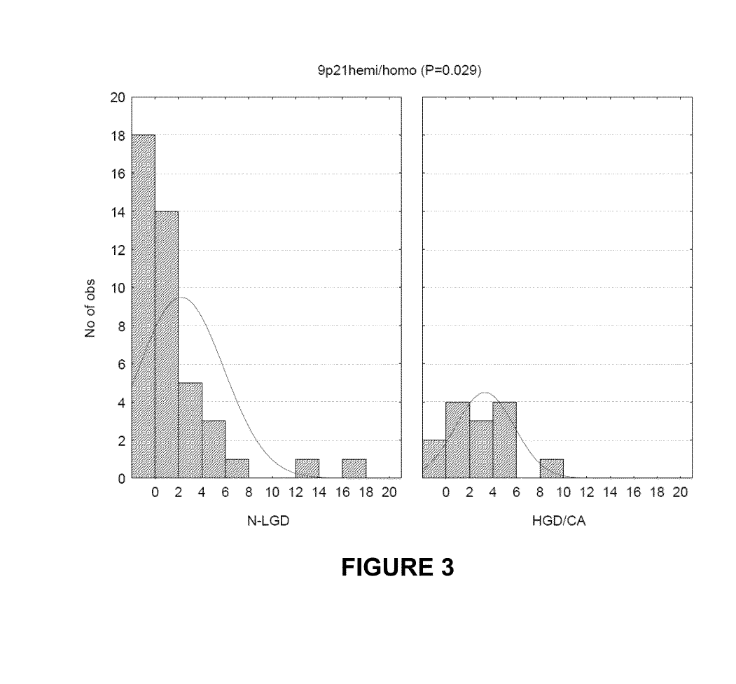

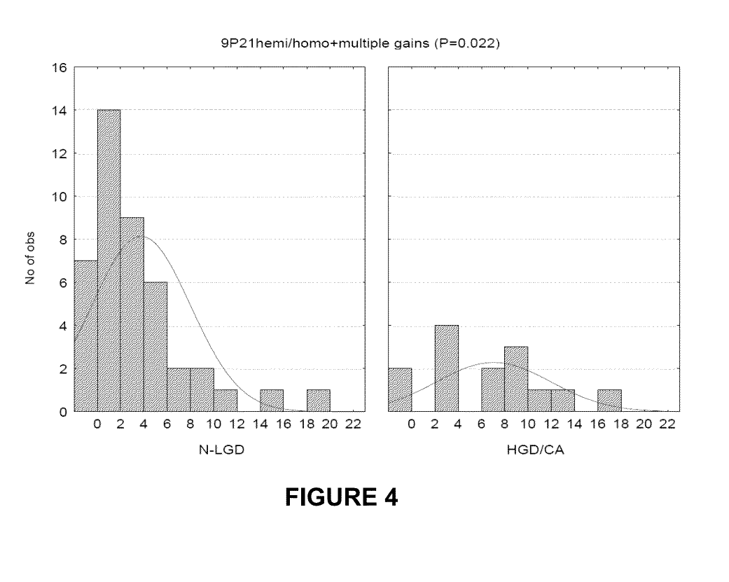

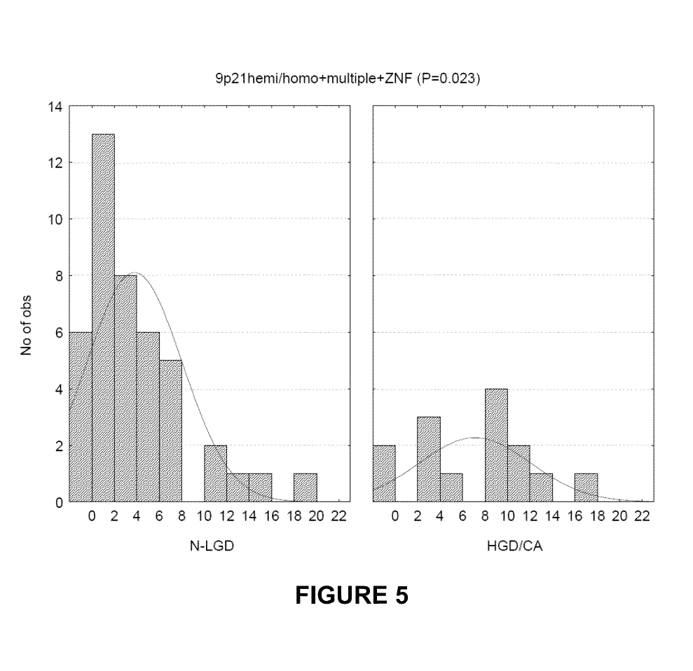

The specimens used for the FISH test consisted of esophageal brushings from 57 patients with Barrett's or suspicious of Barrett's with final diagnosis based on biopsies at the time of brushing or at earlier time. Fourteen (25%) had HGD/CA (2 had CA) and thirteen (30%) of the non HGD/CA had LGD. In this study, data was collected for the following ten variables:
(1-4) Average signals of each of four probes (MYC, 9p21, HER2, and ZNF217) (see, e.g., FIGS. 2A-2F);
(5) Percentage of cells with multiple gains;
(6) Percentage of cells with single gains;
(7) Percentage of cells with 9p21 hemizygous deletion;
(8) Percentage of cells with 9p21 homozygous deletion;
(9) Percentage of cells with tetrasomy; and
(10) Percentage of cells with gains and deletions.

Statistical analysis included assay results compared among groups using Wilcoxon Rank Sum and Fisher exact test, and Receiver Operation Characteristics (ROC).

| Feature | AUC | Pvalue | FDR |
|---|---|---|---|
| Hemizygous 9p21 | 0.71 | 0.01 | 0.11 |
| ZNF217 | 0.69 | 0.02 | 0.08 |
| MultipleGains | 0.69 | 0.03 | 0.06 |
| Homozygous 9p21 | 0.66 | 0.02 | 0.09 |
| HER2 | 0.64 | 0.11 | 0.18 |
| SingleGains | 0.61 | 0.2 | 0.25 |
| GainsNDeletions | 0.6 | 0.1 | 0.21 |
| P16 | 0.54 | 0.66 | 0.74 |
| Tetrasomy | 0.53 | 0.11 | 0.16 |
| MYC | 0.53 | 0.76 | 0.76 |

A “positive” result using the above algorithm indicates a high risk of having High Grade Dysplasia (HGD)/Esophageal adenocarcinoma (EAC). A “negative” result indicates a low risk of having HGD/EAC and suggests normal, reactive, or low grade dysplasia.

The inventive algorithm may be used as part of an ancillary test that can be used to support existing monitoring procedures such as endoscopies and biopsies.

### Example 2

### Pan Brushing of the Esophageal Mucosa

Pan-brushing of the entire esophageal mucosa of patients with BE was performed using a standard well develop protocol. Samples were collected and shipped for testing in PreservCyt fixative.

DNA was isolated from brushing samples using automated DNA extraction method. Sequencing was performed on all brushing samples using ILLUMINA® MISEQ® sequencing system Complete sequencing of the following genes was performed. The corresponding SEQ ID NOs and Genbank Accession Numbers are provided below each gene name, and sequence listings are provided herewith as well as being publicly available from The National Center for Biotechnology Information (NCBI) via the World Wide Web at ncbi.nlm.nih.gov, which information is incorporated herein by reference.

| | | | |
|---|---|---|---|
| ABL1 | EGFR | GNAS | MLH1 |
| (SEQ ID | (SEQ ID | (SEQ ID | (SEQ ID |
| NO. 1) | NO. 2) | NO. 3) | NO. 4) |
| M30833 | M38425 | NM_080426 | FJ940753 |
| RET | AKT1 | ERBB2 | CSF1R |
| (SEQ ID | (SEQ ID | (SEQ ID | (SEQ ID |
| NO. 5) | NO. 6) | NO. 7) | NO. 8) |
| Y12528 | AF283830 | M86910 | M33208 |
| HNF1A | MPL | SMAD4 | ALK |
| (SEQ ID | (SEQ ID | (SEQ ID | (SEQ ID |
| NO. 9) | NO. 10) | NO. 11) | NO. 12) |
| NM_000545 | NM_005373 | AB043547 | U62540 |
| ERBB4 | HRAS | NOTCH1 | GNAQ |
| (SEQ ID | (SEQ ID | (SEQ ID | (SEQ ID |
| NO. 13) | NO. 14) | NO. 15) | NO. 16) |
| L07868 | AF493916 | NM-017617 | U40038 |
| SMARCB1 | APC | FBXW7 | IDH1 |
| (SEQ ID | (SEQ ID | (SEQ ID | (SEQ ID |
| NO. 17) | NO. 18) | NO. 19) | NO. 20) |
| DQ230988 | M74088 | NM_033632 | NM_005896 |
| NPM1 | ATM | FGFR1 | MET |
| (SEQ ID | (SEQ ID | (SEQ ID | (SEQ ID |
| NO. 47) | NO. 21) | NO. 22) | NO. 23) |
| NM_002520 | U82828 | FJ809917 | J02958 |
| JAK2 | BRAF | FGFR2 | JAK3 |
| (SEQ ID | (SEQ ID | (SEQ ID | (SEQ ID |
| NO. 24) | NO. 25) | NO. 26) | NO. 27) |
| NM_004972 | NM_004333 | Z71929 | Y70065 |
| SRC | PDGFRA | STK11 | RB1 |
| (SEQ ID | (SEQ ID | (SEQ ID | (SEQ ID |
| NO. 46) | NO. 28) | NO. 29) | NO. 30) |
| NM_005417 | M21574 | NM_000455 | M33647 |
| CDH1 | FGFR3 | KDR | PIK3CA |
| (SEQ ID | (SEQ ID | (SEQ ID | (SEQ ID |
| NO. 31) | NO. 32) | NO. 33) | NO. 34) |
| L34936 | M58051 | NM_002253 | NM_006218 |
| Z35480 | | | |
| TP53 | CDKN2A | FLT3 | KIT |
| (SEQ ID | (SEQ ID | (SEQ ID | (SEQ ID |
| NO. 35) | NO. 36) | NO. 37) | NO. 38) |
| U94788 | GU086367 | NM_004119 | U63834 |
| | | | X66237 |
| | | | X72595- |
| | | | X72615 |
| PTEN | VHL | PTPN11 | CTNNB1 |
| (SEQ ID | (SEQ ID | (SEQ ID | (SEQ ID |
| NO. 39) | NO. 40) | NO. 41) | NO. 42) |
| AF143312 | AF010238 | EU779708 | AY463360 |
| | U19763 | | |
| | U49746, | | |
| | U68055 | | |
| | U68176 | | |
| KRAS | GNA11 | NRAS | SMO |
| (SEQ ID | (SEQ ID | (SEQ ID | (SEQ ID |
| NO. 43) | NO. 44) | NO. 45) | NO. 48) |
| M54968 | AF493900 | AF493919 | NM_005631 |
| M38506 | | | |

Analysis was performed using multiple filters to avoid overcalling of mutations. Samples were analyzed using 5% and 10% prevalence for the abnormalities. Mutations are considered only when they meet the criteria of being “deleterious” as determined by the SIFT database. Based on analyzing 27 esophageal brushing samples and comparing results with FISH testing and morphologic evaluation of randomly obtained biopsies, clearly our method of brushing and NGS is more sensitive and more objective in detecting EAC.

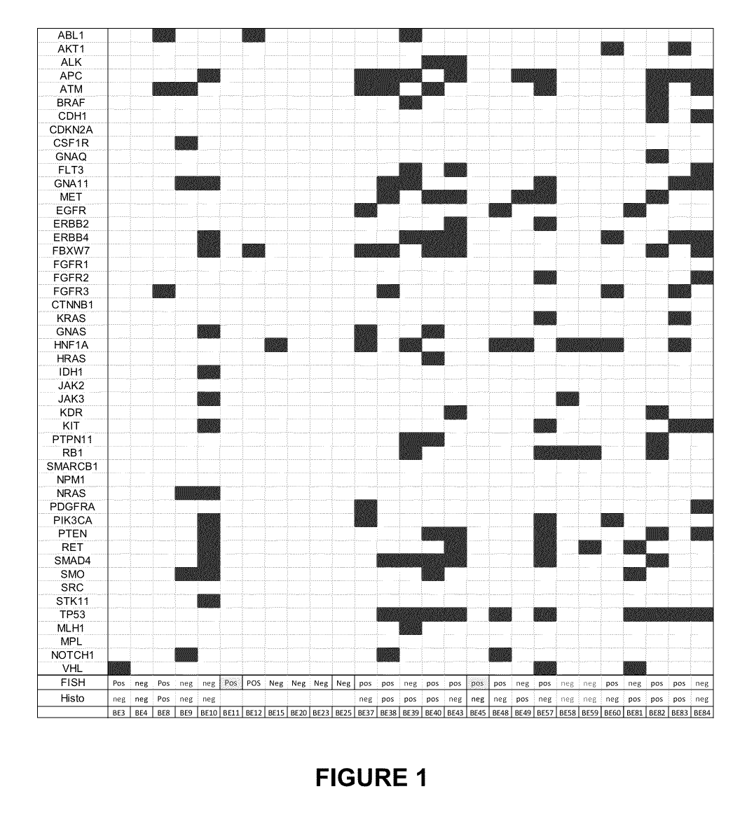

The tile plot of genomic abnormalities (FIG. 1) and the table of the results of the 27 samples (Table 1) show significant genomic abnormalities in 20 of 27 (74%) of testing samples. Notably, 9 of the 21 (43%) cases that had biopsy data were wrongly classified by morphology while only 8 of the 27 cases (30%) showed discrepancy between NGS and FISH, which is more objective than morphology.

TABLE 1

| Sample # | FISH data | Histo Dx | NGS Results |
|---|---|---|---|
| BE3 | Positive | Negative | Positive: VHL |
| BE4 | Negative | Negative | Negative: 0 |
| BE8 | Positive | Positive | Positive: FGFR3, ABL1, ATM |
| BE9 | Negative | Negative | Positive: NRAS, CSF1R, SMO, NOTCH1, ATM |
| BE10 | Negative | Negative | Positive: NRAS, ERBB4, IDH1, PIK3CA, VHL, |
| | | | FBXW7, KIT, APC, SMO, GNAQ, RET, PTPN11, |
| | | | RB1, SMAD4, GNA11, JAK3, STK11, GNAS |
| BE11 | Positive | NA | Negative: 0 |
| BE12 | Positive | NA | Positive: ABL1, FBXW7 |
| BE15 | Negative | NA | Positive: HNF1A |
| BE20 | Negative | NA | Negative: 0 |
| BE23 | Negative | NA | Negative: 0 |
| BE25 | Negative | NA | Negative: 0 |
| BE37 | Positive | Negative | Positive: PIK3CA, PDGFRA, FBXW7, APC, EGFR, |
| | | | HNF1A, GNAS |
| BE38 | Positive | Positive | Positive: FGFR3, FBXW7, APC, MET, NOTCH1, |
| | | | ATM, HNF1A, TP53, SMAD4, GNA11 |
| BE39 | Negative | Positive | Positive: ERBB4, MLH1, KIT, APC, BRAF, ABL1, |
| | | | PTPN11, HNF1A, FLT3, RB1, TP53, SMAD4, |
| | | | GNA11, |
| BE40 | Positive | Positive | Positive: ALK, ERBB4, PIK3CA, FBXW7, MET, |
| | | | SMO, GNAQ, PTEN, ATM, HRAS, PTPN11, ATM, |
| | | | HNF1A, AKT1, TP53, SMAD4, GNAS |
| BE43 | Positive | Negative | Positive: ERBB4, ALK, EGFR3, PDGFRA, FBXW7, |
| | | | KDR, APC, MET, RET, PTEN, FLT3, ERBB2, TP53, |
| | | | SMAD4, |
| BE45 | Positive | Negative | Negative: 0 |
| BE48 | Positive | Negative | Positive: EGFR, NOTCH1, HNF1A, TP53 |
| BE49 | Negative | Positive | Positive: APC, MET, HNF1A, |
| BE57 | Positive | Negative | Positive: PIK3CA, VHL, KIT, APC, NPM1, EGFR, |
| | | | MET, RET, PTEN, FGFR2, ATM, KRAS, HNF1A, |
| | | | RB1, ERBB2, TP53, GNA11 |
| BE58 | Negative | Negative | Positive: HNF1A, RB1, JAK3 |
| BE59 | Negative | Negative | Positive: RET, HNF1A, RB1 |
| BE60 | Positive | Negative | Positive: ERBB4, PIK3CA, FGFR3, HNF1A, AKT1 |
| BE81 | Negative | Positive | Positive: VHL, EGFR, SMO, RET, TP53 |
| BE82 | Positive | Positive | Positive: ERBB4, FBXW7, KDR, APC, BRAF, MET, |
| | | | GNAQ, PTEN, ATM, PTPN11, RB1, CDH1, TP53, |
| | | | SMAD4 |
| BE83 | Positive | Positive | Positive: ALK, ERBB4, FGFR3, KIT, APC, GNAQ, |
| | | | ABL1, KRAS, HNF1A, TP53, GNA11 |
| BE84 | Negative | Negative | Positive: ERBB4, IDH1, FBXW7, KIT, PDGFRA, |
| | | | APC, FGFR2, PTEN, ATM, FLT3, CDH1, |
| | | | TP53, GNA11 |

In summary, combining esophageal brushing and NGS provides a reliable new method for the early detection of EAC. Table 1 provides a comparison of esophageal brushing/NGS with FISH on brushing and biopsies.

1. A method for identifying esophageal adenocarcinoma (EAC) in a subject comprising:
pan-brushing the esophageal mucosa of the subject to obtain a sample;
isolating DNA from the sample;
performing next generation sequencing (NGS) on the isolated DNA; and
analyzing the sequencing results and identifying any genomic abnormalities in one or more driver genes, wherein an abnormality indicates the subject has esophageal adenocarcinoma.

2. The method of claim 1, wherein the one or more driver genes are oncogenes or tumor suppressor genes.

3. The method of claim 1, wherein the one or more driver genes are selected from the group comprising:

| | | | |
|---|---|---|---|
| ABL1 | EGFR | GNAS | MLH1 |
| SEQ ID | SEQ ID | SEQ ID | SEQ ID |
| NO. 1 | NO. 2 | NO. 3 | NO. 4 |
| RET | AKT1 | ERBB2 | CSF1R |
| SEQ ID | SEQ ID | SEQ ID | SEQ ID |
| NO. 5 | NO. 6 | NO. 7 | NO. 8 |
| HNF1A | MPL | SMAD4 | ALK |
| SEQ ID | SEQ ID | SEQ ID | SEQ ID |
| NO. 9 | NO. 10 | NO. 11 | NO. 12 |
| ERBB4 | HRAS | NOTCH1 | GNAQ |
| SEQ ID | SEQ ID | SEQ ID | SEQ ID |
| NO. 13 | NO. 14 | NO. 15 | NO. 16 |
| SMARCB1 | APC | FBXW7 | IDH1 |
| SEQ ID | SEQ ID | SEQ ID | SEQ ID |
| NO. 17 | NO. 18 | NO. 19 | NO. 20 |
| ATM | FGFR1 | MET | JAK2 |
| SEQ ID | SEQ ID | SEQ ID | SEQ ID |
| NO. 21 | NO. 22 | NO. 23 | NO. 24 |
| BRAF | FGFR2 | JAK3 | PDGFRA |
| SEQ ID | SEQ ID | SEQ ID | SEQ ID |
| NO. 25 | NO. 26 | NO. 27 | NO. 28 |
| STK11 | RB1 | CDH1 | FGFR3 |
| SEQ ID | SEQ ID | SEQ ID | SEQ ID |
| NO. 29 | NO. 30 | NO. 31 | NO. 32 |
| KDR | PIK3CA | TP53 | CDKN2A |
| SEQ ID | SEQ ID | SEQ ID | SEQ ID |
| NO. 33 | NO. 34 | NO. 35 | NO. 36 |
| FLT3 | KIT | PTEN | VHL |
| SEQ ID | SEQ ID | SEQ ID | SEQ ID |
| NO. 37 | NO. 38 | NO. 39 | NO. 40 |
| PTPN11 | CTNNB1 | KRAS | GNA11 |
| SEQ ID | SEQ ID | SEQ ID | SEQ ID |
| NO. 41 | NO. 42 | NO. 43 | NO. 44 |
| NRAS | SRC | NPM1 | SMO |
| SEQ ID | SEQ ID | SEQ ID | SEQ ID |
| NO. 45 | NO. 46 | NO. 47 | NO. 48 |

4. The method of claim 1, wherein

| | | | |
|---|---|---|---|
| ABL1 | EGFR | GNAS | MLH1 |
| SEQ ID | SEQ ID | SEQ ID | SEQ ID |
| NO. 1 | NO. 2 | NO. 3 | NO. 4 |
| RET | AKT1 | ERBB2 | CSF1R |
| SEQ ID | SEQ ID | SEQ ID | SEQ ID |
| NO. 5 | NO. 6 | NO. 7 | NO. 8 |
| HNF1A | MPL | SMAD4 | ALK |
| SEQ ID | SEQ ID | SEQ ID | SEQ ID |
| NO. 9 | NO. 10 | NO. 11 | NO. 12 |
| ERBB 4 | HRAS | NOTCH1 | GNAQ |
| SEQ ID | SEQ ID | SEQ ID | SEQ ID |
| NO. 13 | NO. 14 | NO. 15 | NO. 16 |
| SMARCB1 | APC | FBXW7 | IDH1 |
| SEQ ID | SEQ ID | SEQ ID | SEQ ID |
| NO. 17 | NO. 18 | NO. 19 | NO. 20 |
| ATM | FGFR1 | MET | JAK2 |
| SEQ ID | SEQ ID | SEQ ID | SEQ ID |
| NO. 21 | NO. 22 | NO. 23 | NO. 24 |
| BRAF | FGFR2 | JAK3 | PDGFRA |
| SEQ ID | SEQ ID | SEQ ID | SEQ ID |
| NO. 25 | NO. 26 | NO. 27 | NO. 28 |
| STK11 | RB 1 | CDH1 | FGFR3 |
| SEQ ID | SEQ ID | SEQ ID | SEQ ID |
| NO. 29 | NO. 30 | NO. 31 | NO. 32 |
| KDR | PIK3CA | TP53 | CDKN2A |
| SEQ ID | SEQ ID | SEQ ID | SEQ ID |
| NO. 33 | NO. 34 | NO. 35 | NO. 36 |
| FLT3 | KIT | PTEN | VHL |
| SEQ ID | SEQ ID | SEQ ID | SEQ ID |
| NO. 37 | NO. 38 | NO. 39 | NO. 40 |
| PTPN11 | CTNNB1 | KRAS | GNA11 |
| SEQ ID | SEQ ID | SEQ ID | SEQ ID |
| NO. 41 | NO. 42 | NO. 43 | NO. 44 |
| NRAS | SRC | NPM1 | SMO |
| SEQ ID | SEQ ID | SEQ ID | SEQ ID |
| NO. 45 | NO. 46 | NO. 47 | NO. 48 |

are completely sequenced and analyzed

5. The method of claim 1, wherein an abnormality in at least one gene indicates the subject has esophageal adenocarcinoma.

6. The method of claim 1, wherein the entire esophageal mucosa is pan-brushed.

7. The method of claim 1, wherein the sequencing is step is performed using ILLUMINA® MISEQ® sequencer, or an equivalent thereto.

8. The method of claim 1, wherein the analysis is performed using multiple filters.

9. The method of claim 1, wherein 5% prevalence for the abnormality indicates the subject has esophageal adenocarcinoma.

10. The method of claim 1, wherein 10% prevalence for the abnormality indicates the subject has esophageal adenocarcinoma.

11. A method for determining the risk of esophageal adenocarcinoma (EAC) in a subject, comprising:
performing fluorescence in situ hybridization (FISH) testing on esophageal biopsy samples taken from a patient known or suspected to have Barrett's esophagus (BE);
generating values corresponding to each of a plurality of data points obtained during FISH testing, the plurality of data points including data selected from the group consisting of average signals of each of four probes (MYC (SEQ ID NO. 51), 9p21 (SEQ ID NO. 49), HER2 (SEQ ID NO. 50), and ZNF217 (SEQ ID NO. 52)), percentage of cells with multiple gains, percentage of cells with single gains, percentage of cells with 9p21 hemizygous deletion, percentage of cells with 9p21 homozygous deletion, percentage of cells with tetrasomy, and percentage of cells with gains and deletions;
applying an algorithm to the values to generate a numerical score;
comparing the numerical score to one or more thresholds, wherein the threshold defines a distinction between HGD/EC and normal or non-HGD/EC.

12. The method of claim 11, wherein the one or more thresholds comprise:

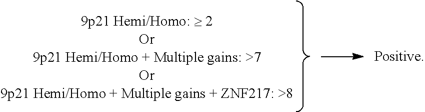

13. A method for morphologic evaluation of BE histology, comprising:
performing fluorescence in situ hybridization (FISH) testing on esophageal biopsy samples taken from a patient known or suspected to have Barrett's esophagus (BE);
generating values corresponding to each of a plurality of data points obtained during FISH testing, the plurality of data points including data selected from the group consisting of average signals of each of four probes (MYC (SEQ ID NO. 51), 9p21 (SEQ ID NO. 49), HER2 (SEQ ID NO. 50), and ZNF217 (SEQ ID NO. 52)), percentage of cells with multiple gains, percentage of cells with single gains, percentage of cells with 9p21 hemizygous deletion, percentage of cells with 9p21 homozygous deletion, percentage of cells with tetrasomy, and percentage of cells with gains and deletions;
applying an algorithm to the values to generate a numerical score;
comparing the numerical score to one or more thresholds, wherein the threshold defines a distinction between HGD/EC and normal or non-HGD/EC.

14. The method of claim 13, wherein the one or more thresholds comprise:

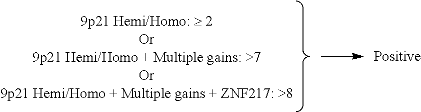

15. A method for determining the prognosis of BE, comprising:
performing fluorescence in situ hybridization (FISH) testing on esophageal biopsy samples taken from a patient known or suspected to have Barrett's esophagus (BE);
generating values corresponding to each of a plurality of data points obtained during FISH testing, the plurality of data points including data selected from the group consisting of average signals of each of four probes (MYC (SEQ ID NO. 51), 9p21 (SEQ ID NO. 49), HER2 (SEQ ID NO. 50), and ZNF217 (SEQ ID NO. 52)), percentage of cells with multiple gains, percentage of cells with single gains, percentage of cells with 9p21 hemizygous deletion, percentage of cells with 9p21 homozygous deletion, percentage of cells with tetrasomy, and percentage of cells with gains and deletions;
applying an algorithm to the values to generate a numerical score;
comparing the numerical score to one or more thresholds, wherein the threshold defines a distinction between HGD/EC and normal or non-HGD/EC.

16. The method of claim 15, wherein the one or more thresholds comprise: [chemical structure]
